(19) United States
(12) Patent Application Publication
(10) Pub. No.: US 2013/0183279 A1
(43) Pub. Date: Jul. 18, 2013

(54) Fibrin Formulations for Wound Healing

(71) Applicant: Manuela Mûller--Maissen, Zurich (CH)

(72) Inventor: Manuela Mûller--Maissen, Zurich (CH)

(73) Assignee: Kuros Biosurgery AG

(21) Appl. No.: 13/555,991

(22) Filed: Jul. 23, 2012

Related U.S. Application Data
(63) Continuation-in-part of application No. 12/342,420, filed on Dec. 23, 2008, now Pat. No. 8,226,942.
Application No. 12/342,420 is a continuation of application No. PCT/EP2008/068185, filed on Dec. 22, 2008.
(60) Provisional application No. 61/017,409, filed on Dec. 28, 2007.

(51) Int. Cl. A61K 38/36; A61K 38/54; A61K 38/30; A61K 38/48; A61K 38/18

(52) U.S. Cl. CPC A61K 38/363; A61K 38/48; A61K 38/1858; A61K 38/1841; A61K 38/1866; A61K 38/1825; A61K 38/30; A61K 38/54
USPC 424/94.3; 424/94.64

(57) ABSTRACT

Fibrin formulations, fibrin matrices and kits for wound healing, use of the formulation, matrices and foams, and kits and methods of using thereof, are described herein. In a preferred aspect, the compositions are suitable for use for local administration. In another preferred aspect, the compositions are also suitable for use in methods for forming enhanced controlled delivery fibrin matrices and foams.

### CROSS REFERENCE TO RELATED APPLICATION

This application is a continuation-in-part of U.S. Ser. No. 12/342,420, filed Dec. 23, 2008, which is a continuation of PCT/EP2008/068185 filed on Dec. 22, 2008, which claims priority to U.S. Provisional Application No. 61/017,409, filed on Dec. 28, 2007; the disclosures of these applications are incorporated herein by reference.

### REFERENCE TO SEQUENCE LISTING

The Sequence Listing submitted Jul. 23, 2012 as a text file named “KUROS_{—}138_CIP_ST25.txt,” created on Jul. 23, 2012, and having a size of 3,000 bytes is hereby incorporated by reference.

### FIELD OF THE INVENTION

The present invention generally relates to fibrin compositions, fibrin formulations, kits and methods for forming fibrin matrices or foams which optionally can include bioactive factors like proteins and thus form supplemented matrices or foams. These fibrin matrices and foams, which optionally can be supplemented, are useful in tissue repair and regeneration of wounds, in particular of skin wounds. A preferred protein is a platelet derived growth factor which can be in the form of a fusion protein comprising a transglutaminase substrate domain which enables the covalently linked to the fibrin matrix or foam. The present invention also relates to methods for forming fibrin formulations, matrices or foams optionally supplemented.

### BACKGROUND OF THE INVENTION

For tissue repair or regeneration, cells must migrate into a wound bed, proliferate, express matrix components or form extracellular matrix, and form a final tissue shape. Multiple cell populations must often participate in this morphogenetic response, frequently including vascular and nerve cells. Matrices have been demonstrated to greatly enhance, and in some cases have been found to be essential, for this to occur. Natural matrices are subject to remodelling by cellular influences, all based on proteolysis, e.g., by plasmin (degrading fibrin) and matrix metalloproteinases (degrading collagen, elastin, etc.). Such degradation is highly localized, and only upon direct contact with the cell. If the matrix contains bioactive factors, such as growth factors, the cellular influences can tightly regulate the delivery of these substances from the matrix.

When a tissue is injured, polypeptide growth factors which exhibit an array of biological activities are released by the body into the wound where they play a crucial role in healing (see, e.g., Hormonal Proteins and Peptides, Li, C. H., ed., Volume 7, Academic Press, Inc. New York, pp. 231-277 and Brunt et al., Biotechnology 6:25-30 (1988)). These activities include, recruiting cells, such as leukocytes and fibroblasts, into the injured area, and inducing cell proliferation and differentiation. Growth factors that participate in wound healing include: platelet-derived growth factor (PDGF), insulin-binding growth factor-1 (IGF-1), insulin-binding growth factor-2 (IGF-2), epidermal growth factor (EGF), transforming growth factor-α (TGF-α), transforming growth factor-β (TGF-β), platelet factor 4 (PF-4), and heparin binding growth factors one and two (HBGF-1 and HBGF-2).

Fibrin is a natural, blood derived material which has been reported for several biomedical applications. Fibrin matrices have been used as sealants due to their ability to adhere to many tissues and their natural role in wound healing. Some specific applications include use as a sealant for vascular graft attachment, heart valve attachment, bone positioning in fractures, tendon repair and for neuronal regeneration. Additionally, these matrices have been used as drug delivery devices as well as material for cell in-growth scaffolds or matrices (U.S. Pat. No. 6,331,422 to Hubbell et al.).

The commercially available fibrin sealants bear—as a human blood derived product—the inherent risk of adverse reaction of the patient to its non-autologous components, which might include allergic and hypersensitive reactions. This becomes the more relevant the larger the amount of fibrin sealant is which have to be applied to the site of need and/or if the fibrin sealant contains additional added bioactive factors.

The incorporation of bioactive factors, like growth factors, in biomaterials can be achieved by incorporating the bioactive factor through physical interaction with the formulation and matrix and has been described, for example, in U.S. Pat. No. 6,117,425; U.S. Pat. No. 6,197,325; and WO02/085422. Covalent linking of the bioactive factor to the biomaterial is a more advanced technique allowing improved control of the release profile of the bioactive factor from the biomaterial. The incorporation of small synthetic or naturally occurring molecules, peptides and/or proteins into fibrin matrices through action of transglutaminases has been described in U.S. Pat. Nos. 6,331,422; 6,468,731 and 6,960,452 and WO 03/052091 and Schense, et al., Bioconj. Chem., 10:75-81 (1999). Covalent cross-linking of the bioactive factor may be performed by modifying the bioactive factor through incorporation of functional groups, which are able to react with one or more of the reactive groups of the precursor components or biomaterials during or after formation of the biomaterial. U.S. patent application No 2003/0187232 discloses a fibrin matrix supplemented with a PDGF modified with transglutaminase substrate domain and its use in chronic wound healing in human patients. However, with the system described therein, a high amount of growth factor is released from the fibrin gel in the first hours after application.

It is an object of the invention to provide a fibrin formulation and matrix with a decreased risk of adverse reactions in the patient while maintaining or improving its performance for a given indication, like adhesion properties, manipulation time etc.

It is another object of the invention to provide a fibrin formulation and matrix which shows adhesion and healing properties. This is necessary e.g. when a first layer of tissue (or other material) is placed upon a second layer of tissue and the fibrin matrix does not only adhere the two layers together but also increase the quality of healing and/or time to healing of the two layers, i.e. the time and/or quality of two layers growing together. It is a further object of the present invention to provide a fibrin formulation, optionally supplemented with bioactive factors, exhibiting adhesiveness to the underlying tissue even when the application site is not horizontally located and when the formulation reaching its gel state only after extended periods.

It is a further object of the present invention to provide fibrin formulations and matrices for enhanced controlled and/or sustained release of growth factors.

It is a further object of the present invention to provide compositions, formulations and methods for the formation of a fibrin foam which can be supplemented with growth factors.

### SUMMARY OF THE INVENTION

Fibrin Compositions, fibrin formulations for wound treatment use of the composition and formulation, kits and methods of preparation and using thereof are described herein.

In one aspect the fibrin compositions and fibrin formulation are suitable for forming fibrin matrices which have a decreased risk of causing adverse reactions in a patient's body while maintaining its adhesive properties.

In another aspect the fibrin compositions are suitable of forming fibrin formulations and matrices which show adhesive and wound healing properties.

In another aspect, the fibrin compositions and formulations are suitable for forming a fibrin foam that can be applied at the site of need without the risk of and which stays to the application site.

In another aspect, the compositions are suitable for forming fibrin matrices with enhanced controlled release of bioactive factors incorporated therein.

In another preferred aspect, the compositions are also suitable for use in methods for forming controlled delivery fibrin matrices that can be administered as foams.

Fibrin formulations are provided that comprise
(i) fibrinogen and
(ii) thrombin wherein the amount of thrombin is less than 0.3 UI of thrombin/mg of fibrinogen.

The formulation can further comprise a calcium source.

The fibrinogen concentration of the fibrin composition of the present invention is in a range of about 10 mg to about 130 mg per ml of fibrinogen precursor solution. In a preferred embodiment of the present invention the fibrinogen precursor solution and the thrombin precursor solution is mixed in a volume ratio of 1:1 for forming the fibrin formulation. Thus the fibrinogen concentration is in a range of 5 mg to 65 mg per ml of fibrin formulation, preferably in a range of between 7.5 to 30 mg fibrinogen per ml fibrin formulation, more preferably in a range of between 10 to 29 mg per ml fibrin formulation and most preferred in a range of between 15 to 27.5 mg fibrinogen per ml fibrin formulation. The above mentioned preferred concentration ranges of fibrinogen provide a fibrin formulation and—following crosslinking of the fibrinogen monomers—a fibrin matrix which has a substantially decreased concentration of fibrinogen compared to the fibrin sealants on the market. The decreased concentration of the non autologous protein fibrinogen decreases the risk of adverse reactions in the patients.

The thrombin concentration in the fibrin formulation is preferably from about 0.015 to about 0.29 I.U. thrombin per mg of fibrinogen, more preferably from about 0.04 to about 0,28 I.U. thrombin per mg of fibrinogen, most preferably about 0.08 LU. thrombin per mg of fibrinogen.

The fibrin formulation can be foamed by additionally comprising a biocompatible gas selected from the group consisting of CO2, N2, air and an inert gas in an effective amount to form the desired foamed fibrin formulation which will result in a fibrin foam following crosslinking of the fibrinogen.

A particularly preferred embodiment of the present invention is thus a biocompatible gas containing fibrin formulation that comprises
(i) fibrinogen in a range of between 7.5 to 30 mg fibrinogen per ml of fibrin formulation, preferably in between 10 to 29 mg per ml fibrin formulation and most preferred in between 15 to 27.5 mg fibrinogen per ml fibrin formulation;
(ii) thrombin wherein the amount of thrombin is less than 0.3 I.U. thrombin/mg of fibrinogen, preferably in a range of between 0.015 to 0.29 I.U. thrombin/mg of fibrinogen, more preferably from about 0.04 to 0.28 I.U. thrombin/mg of fibrinogen
(iii) biocompatible gas, in particular air.

The fibrin formulation can further comprise a calcium source. The fibrinogen in this gas-containing fibrin formulation crosslinks over time to form a fibrin foam which shows favourable responses in the treatment of chronic skin wounds in particular in diabetic chronic ulcers.

The fibrin formulation of the present invention can further comprise added bioactive factors in a concentration range of between about 1 and about 20 i.g per mg of fibrinogen, preferably from about 1.32 to about 16 μg per mg of fibrinogen and most preferably from 4 to about 12 μg per mg of fibrinogen. These concentration ranges are suitable for chronic skin wounds caused by diabetes, circulation problems or extended bed rests due to illness or operation (pressure sores).

Certain indications however require much lower amounts of added bioactive factor to achieve satisfying healing results and to avoid unwanted effects, like hypergranulation and necrosis of skin grafts. For these indications the added bioactive factor is in a range of between 1.5 μg to 0.0001 μg added bioactive factor per mg of fibrinogen.

Another preferred embodiment of the present invention is thus a fibrin formulation including:

(i) fibrinogen;

(ii) thrombin wherein the amount of thrombin is less than 0.3 UI of thrombin per mg of fibrinogen; and

(iii) added bioactive factor in a concentration range of between about 0.0001 μg about 15 μg bioactive factor per mg of fibrinogen.

More preferred the bioactive factor is added in a concentration range of between about 0.0002 μg and about 0.8 μg added bioactive factor per mg of fibrinogen and most preferred a range of between about 0.0004 μg and about 0.5 μg added bioactive factor per mg fibrinogen. Fibrin formulations with these low concentrations of bioactive factors are in particular suitable for the treatment of acute wounds, e.g. skin grafting procedures or various kind of flap surgeries which require certain parts of soft and muscle tissue to adhere and grow together following separation of these layers and their manipulation during operation. One example of such a procedure is abdominoplasty, in which seroma formation is a quite common complication. The fibrin formulations and matrices of the present invention are e.g. designed to reduce complications due to seroma formation. The added bioactive factors are preferably growth factors which are members of the transforming growth factor (TGF β) superfamily and members of the platelet derived growth factor (PDGF) and (FGF) superfamily. In particular, preferred members are PFGF, TGFβ, BMP, VEGF, FGF and Insulin-like growth factor (IGF) and most preferred are PDGF AB, PDGF BB, PDGF D, TGFβ1, TGFβ3, VEGF 121, FGF 7 and IGF 1. In a preferred embodiment the growth factor is PDGF AB.

Various bioactive factors can be combined however preferably only one bioactive factor, preferably PDGF AB, is added to the fibrin formulation of the present invention. In another preferred embodiment of the present invention the bioactive factor is provided as a fusion protein which has the bioactive factor, preferably PDGF AB, in one domain and a transglutaminase substrate domain in a second domain. The transglutaminase substrate domain is able to covalently crosslink to the fibrin matrix during its crosslinking. In one embodiment, the transglutaminase substrate domain (TG) is a Factor XIIIa substrate domain. Preferably, the Factor XIIIa substrate domain comprises SEQ ID NO:1.

The fusion protein further can optionally include a degradation site between the first and the second domain of the fusion protein. In a preferred embodiment, the degradation site is an enzymatic or hydrolytic degradation site. In a most preferred embodiment, the degradation site is an enzymatic degradation site, which is cleaved by an enzyme selected from the group consisting of plasmin and matrix metalloproteinase.

In a most preferred embodiment, the fusion protein comprises an amino acid sequence of SEQ ID NO:2 and SEQ ID NO:3, The fusion proteins is incorporated in such a way that the protein is covalently linked to the matrix, retains its biological activity and is slowly released in the first hours following application.

The fibrinogen concentration is in a range of 5 mg to 65 mg per ml of fibrin formulation, preferably in a range of between 7.5 to 30 mg fibrinogen per ml fibrin formulation, more preferably in a range of between 10 to 29 mg per ml fibrin formulation and most preferred in a range of between 15 to 27.5 mg fibrinogen per ml fibrin formulation. The fibrin formulation can further comprise a calcium source. This preferred fibrin formulation (and—following crosslinking of the fibrinogen fibrin matrix) maintains their adhesion and healing properties even when the concentration of fibrinogen is lower than 65 mg per ml of fibrin formulation to ranges as mentioned hereinbefore.

In a preferred embodiment, the thrombin concentration is from about 0.015 to about 0.29I.U. thrombin per mg of fibrinogen, more preferably from about 0.04 to about 0.28 I.U. thrombin per mg of fibrinogen, most preferably about 0.08 I.U. thrombin per mg of fibrinogen.

A further aspect of the invention is a kit suitable in the formation of a fibrin formulation, said kit includes

(i) a first container comprising fibrinogen; and

(ii) a second container comprising thrombin, wherein the amount of thrombin is less than 0.3 U.I. thrombin per mg of fibrinogen; and optionally a calcium source.

The fibrinogen concentration is in a range of between 5 mg to 65 mg per ml fibrin formulation formed by the kit, preferably in a range of between 7.5 to 30 mg fibrinogen per ml fibrin formulation, more preferably in a range of between 10 to 29 mg per ml fibrin formulation and most preferred in a range of between 15 to 27.5 mg fibrinogen per ml fibrin composition. Assuming a volume ratio of 1:1 of the thrombin and fibrinogen precursor solution in the kit, a fibrinogen concentration of between 5 mg to 65 mg per ml of fibrin formulation can be obtained by a fibrinogen concentration in a range of about 10 mg to 130 mg per ml of fibrinogen precursor solution, and the lower concentrations by 15 to 60 mg of fibrinogen per ml fibrinogen precursor solution and 20 to 58 mg per ml fibrin precursor component. However there are also other ways of achieving a fibrinogen concentration, like having the fibrinogen as a lyophilized powder and reconstitution of the lyophilized fibrinogen in the thrombin precursor component.

The thrombin concentration is preferably from about 0.015 to 0.29 I.U. thrombin per mg of fibrinogen, more preferably from about 0.04 to 0.28 I.U. thrombin per mg of fibrinogen, preferably about 0.08 I.U. thrombin per mg of fibrinogen.

The kit of the present invention can further comprise a biocompatible gas selected from the group consisting of CO₂, N₂, air or an inert gas, preferably air. The biocompatible gas can be part of the contents of the first and/or the second container or in a third container separate from the first and second container.

The kit of the present invention can further comprise added bioactive factors in a concentration range of between 1 to 20 μg per mg of fibrinogen, preferably from about 1.32 to 16 μg per mg of fibrinogen and most preferably from 4 to 12 μg per mg of fibrinogen. The added bioactive factors can be part of the first and/or second container or can be in a separate third container to be mixed with the first and second container upon formation of the fibrin composition. Theses concentration ranges are suitable e.g. for chronic skin wounds.

In the kits for certain other indications like acute wounds the added bioactive factors are in a concentration range of between 1.5 μg to 0.0001 μg added bioactive factor per mg of fibrinogen. More preferred is a range of between 0.8 μg to 0.0002 μg added bioactive factor per mg of fibrinogen and most preferred a range of between 0.5 μg to 0.0004 μg added bioactive factor per mg fibrinogen. The bioactive factors are preferably growth factors which are members of the transforming growth factor (TGF β) superfamily and members of the platelet derived growth factor (PDGF) and (FGF) super-family. In particular, preferred members are PDGF, TGFβ, BMP, VEGF, FGF and Insulin-like growth factor (IGF) and most preferred are PDGF AB, PDGF BB, PDGF D, TGFβ1, TGFβ3, VEGF 121, FGF 7 and IGF 1. In a preferred embodiment the growth factor is PDGF AB as the only added growth factor. In a most preferred embodiment the bioactive factor is provided as a fusion protein which has the bioactive factor, preferably PDGF AB, in one domain and a transglutaminase substrate domain in a second domain. The transglutaminase substrate domain is able to covalently crosslink to the fibrin matrix during its formation.

Thus, in further aspect, the invention provides a kit including

(i) a first container comprising fibrinogen and at least one fusion protein, comprising a first domain comprising a PDGF and a second domain comprising a substrate domain for a crosslinking enzyme; and

(ii) a second container comprising thrombin, wherein the amount of thrombin is less than 0.3 U.I. thrombin per mg of fibrinogen; and a calcium source.

The kit of the present invention can further comprise a biocompatible gas selected from the group consisting of CO₂, N₂, air or an inert gas, preferably air. The biocompatible gas is either in the first or the second container.

In further aspect, the invention provides a method for preparing a fibrin formulation, the method comprising the steps of
(iii) providing a fibrinogen solution; and
(iv) providing a thrombin solution, wherein the amount of thrombin is less than 0.3 U.I. thrombin per mg of fibrinogen;
(v) optionally providing a calcium source.

The fibrinogen concentration is in a range of 5 mg to 65 mg per ml of fibrin formulation, preferably in a range of between 7.5 to 30 mg fibrinogen per ml fibrin formulation, more preferably in a range of between 10 to 29 mg per ml fibrin formulation and most preferred in a range of between 15 to 27.5 mg fibrinogen per ml fibrin formulation.

The thrombin concentration is preferably from about 0.015 to 0.29 I.U. thrombin per mg of fibrinogen, more preferably from about 0.04 to 0.28 I.U. thrombin per mg of fibrinogen, preferably about 0.08 I.U. thrombin per mg of fibrinogen.

The method of the present invention can further include the step of providing a biocompatible gas selected from the group consisting of CO₂, N₂, air or an inert gas, preferably air. Preferably the volume of the biocompatible gas is between 80 and 120% of the volume of the fibrin formulation, preferably 100%. The biocompatible gas can be part of the fibrinogen and/or thrombin precursor solution and mixed at the same time the fibrinogen and thrombin precursor solutions are mixed with each other or can be provided in a third separate container and either mixed while mixing the fibrinogen and thrombin part or immediately after mixing the fibrinogen and thrombin precursor components.

The method of the present invention can further comprise providing added bioactive factors in a concentration range of between 1 to 20 μg per mg of fibrinogen, preferably from about 1.32 to 16 μg per mg of fibrinogen and most preferably from 4 to 12 μg per mg of fibrinogen and mixing the fibrinogen solution, thrombin solution and bioactive factor to form a supplemented fibrin formulation. The added bioactive factors can be part of the first and/or second container or can be in a separate third container to be mixed with the content of the first and/or second container before or after mixing the fibrinogen and thrombin solution to form the fibrin formulation. For certain other indications the added bioactive factor is in a concentration range of between 1.5 μg to 0.0001 μg bioactive factor per mg of fibrinogen. More preferred is a range of between 0.8 μg to 0.0002 μg bioactive factor per mg of fibrinogen and most preferred a range of between 0.5 μg to 0.0004 μg bioactive factor per mg fibrinogen. The bioactive factors are preferably growth factors which are members of the transforming growth factor (TGF β) superfamily and members of the platelet derived growth factor (PDGF) and (FGF) superfamily. In particular, preferred members are PDGF, TGFβ, BMP, VEGF, FGF and Insulin-like growth factor (IGF) and most preferred are PDGF AB, PDGF BB, PDGF D, TGFβ1, TGFβ3, VEGF 121, FGF 7 and IGF 1. In a preferred embodiment the growth factor is PDGF AB as the only added growth factor . In a most preferred embodiment the bioactive factor is provided as a fusion protein which has the bioactive factor, preferably PDGF AB, in one domain and a transglutaminase substrate domain in a second domain. The transglutaminase substrate domain is able to covalently crosslink to the fibrin matrix during its formation. In one embodiment, the transglutaminase substrate domain is a factor XIIa substrate domain.

In a further aspect, the present invention provides a method for preparing a fibrin matrix having at least one fusion protein, the method including the steps of:

(i) providing a fibrinogen solution;

(ii) providing a thrombin solution wherein the amount of thrombin is less than 0.3 I.U. thrombin per mg of fibrinogen;

(iii) providing at least one fusion protein comprising a first domain comprising a PDGF and a second domain comprising a transglutaminase substrate domain; and

(iv) mixing components provided in steps (i), (ii) and (iii) to crosslink the matrix material such that the fusion protein is covalently linked to the matrix through the second domain.

In order to form a fibrin foam, components provided in steps (i), (ii) and (iii) are mixed with a biocompatible gas selected from the group consisting of CO₂, N₂, air or an inert gas, preferably air to crosslink the foam material such that the fusion protein is covalently linked to the matrix through the second domain.

In a preferred embodiment, the volume of the biocompatible gas is from about 80 to 120% of the volume of fibrin formulation, preferably about 100%.

A further aspect provides a fibrin matrix obtained according to the disclosed method. Preferably, a controlled delivery fibrin matric is obtained. In a particular preferred embodiment no more than 25% of the added bioactive factor or growth factor is released after incubation of the controlled delivery fibrin matrix during 3 days at 37° C. in a buffer solution.

Still another embodiment provides a controlled delivery fibrin foam obtained according to the disclosed methods. Preferably, no more than 25% of the growth factor is released after incubation of the controlled delivery fibrin matrix for 3 days at 37° C. in a buffer solution.

Another aspect provides a fibrin foam including:
(i) fibrinogen;
(ii) thrombin wherein the amount of thrombin is less than 0.3 I.U. of thrombin/mg of fibrinogen; and
(iii) optionally at least one added bioactive factor, r, and
(iv) a biocompatible gas selected from the group consisting of CO₂, N₂, air or an inert gas, preferably air.

The added bioactive factor is preferably PDGF AB and even more preferably a fusion protein comprising a first domain comprising a PDGF and a second domain comprising a transglutaminase substrate domain. The fibrinogen concentration is in a range of 5 mg to 65 mg per ml of fibrin formulation, preferably in a range of between 7.5 to 30 mg fibrinogen per ml fibrin formulation, more preferably in a range of between 10 to 29 mg per ml fibrin formulation and most preferred in a range of between 15 to 27.5 mg fibrinogen per ml fibrin formulation.

The thrombin concentration is preferably from about 0.015 to 0.29 I.U.

thrombin per mg of fibrinogen, more preferably from about 0.04 to 0.28 I.U. thrombin per mg of fibrinogen, preferably about 0.08 I.U. thrombin per mg of fibrinogen.

In another embodiment, the biocompatible gas is selected from the group consisting of CO₂, N₂, air or an inert gas such as Freon and is preferably air.

Still other embodiments include: fibrin formulations and/or fibrin foams for use as a in treatment of chronic wounds, preferably diabetic ulcers; controlled delivery fibrin matrices or foams for use as a medicament; controlled delivery fibrin matrices or foams for use in treatment of a wound, preferably wherein the wound is an ulcer caused by diabetes; and the use of the fibrin foams or controlled delivery fibrin matrices or fibrin foams o for the manufacture of a medicament for treatment of a wound, preferably wherein the wound is an ulcer caused by diabetes.

### BRIEF DESCRIPTION OF THE DRAWINGS

FIG. 1 is a line graph of the percent release of TG-PDGF.AB versus time (hours) from a fibrin matrix prepared with 50 mg/ml of fibrinogen, 250 I.U./ml of thrombin and 600 μg/ml of TG-PDGF.AB. Five experiments are plotted on the graph.

FIG. 2 is a line graph of the percent release of TG-PDGF.AB versus time (hours) from a fibrin matrix prepared with 50 mg/ml of fibrinogen, 250 I.U./ml of thrombin and 66 μg/ml of TG-PDGF.AB. Five experiments are plotted on the graph.

FIG. 3 is a line graph of the percent release of TG-PDGF.AB versus time (hours) from a fibrin matrix prepared with 50 mg/ml of fibrinogen, 600 μg/ml of TG-PDGF.AB and 4 I.U./ml (□), 15 I.U./ml (×), 31 I.U./ml (▴), 62 I.U./ml (▪), 125 I.U./ml (♦) and 250 I.U./ml () of thrombin.

FIG. 4 is a line graph of the percent release of TG-PDGF.AB versus time (hours) from a fibrin matrix prepared with 50 mg/ml of fibrinogen, 66 μg/ml of TG-PDGF.AB and 4 I.U./ml (), 15 I.U./ml (), 31 I.U./ml (Δ), 62 I.U./ml (▴), 125 I.U./ml (▪) and 250 I.U./ml (♦) of thrombin.

FIG. 5 is a line graph of the percent release of TG-PDGF.AB versus time (hours) from a fibrin matrix prepared with 50 mg/ml of fibrinogen, and 66 μg/ml (final concentration of 33 μg/ml) of TG-PDGF.AB and 4 I.U./ml and 250 I.U./ml of thrombin and a fibrin matrix prepared with 50 mg/ml of fibrinogen, 600 μg/ml of TG-PDGRAB (final concentration 300 μg/ml) and 4 I.U./ml and 250 I.U./ml of thrombin.

FIG. 6 is a line graph of the percent release of TG-PDGF.AB versus time (hours) from a fibrin matrix prepared with 50 mg/ml of fibrinogen, 600 μg/ml of TG-PDGF.AB and 250 I.U./ml of thrombin with factor XIII concentration of 0 I.U./ml (♦), 0.1 I.U./ml (▪), 1 I.U./ml (▴) and 10 I.U./ml (×).

FIG. 7 is a line graph of the percent release of TG-PDGF.AB versus time (hours) from a fibrin matrix prepared with 50 mg/ml of fibrinogen, 66 μg/ml of TG-PDGF.AB and 250 I.U./ml of thrombin with factor XIII concentration of 0 I.U./ml (♦), 0.1 I.U./ml (▪), 1 I.U./ml (▴) and 10 I.U./ml (×).

FIG. 8 is a line graph of the percent release of TG-PDGF.AB versus time (hours) from a fibrin matrix prepared with 50 mg/ml of fibrinogen, 600 μg/ml of TG-PDGF.AB and 4 I.U./ml of thrombin with factor XIII concentration of 0 I.U./ml (♦), 0.1 I.U./ml (▪), 1 I.U./ml (▴) and 10 I.U./ml (×).

FIG. 9 is a line graph of the percent release of TG-PDGF.AB versus time (hours) from a fibrin matrix prepared with 50 mg/ml of fibrinogen, 66 μg/ml of TG-PDGF.AB and 4 I.U./ml of thrombin with factor XIII concentration of 0 I.U./m1(♦), 0.1 I.U./ml (▪), 1 I.U./ml (▴) and 10 I.U./ml (×).

FIG. 10 is a release comparison of TG-PDGF.AB from test items incubated in buffer until full degradation (buffer changed (♦)) or over 14 days without degradation (buffer not changed (▪)).

FIG. 11 is the release profile (% TG-PDGF.AB released vs. time) of TG-PDGF.AB and native PDGF-AB from fibrin foam clots for three concentrations (High, Middle, Low doses). Native PDGF-AB low dose (♦),Native PDGF-AB middle dose (▪) ,Native PDGF-AB high dose (▴) TG-PDGF.AB low dose (×), TG-PDGF.AB middle dose (□) and TG-PDGF.AB high dose (−).

### DETAILED DESCRIPTION OF THE INVENTION

### I. Definitions

“Added bioactive factor” as used herein means a bioactive factor that is not present in the precursor composition, fibrin formulation and/or fibrin matrix, but is added to the precursor composition and/or fibrin formulation so that it is incorporated into the resulting fibrin matrix. Bioactive factors include peptides, proteins, and polysaccharides, and are preferably a growth factor or hormone.

“Matrix” as generally used herein refers to a material intended to interface with biological systems to treat, augment, or replace any tissue or function of the tissue depending on the material either permanently or temporarily. The matrix can serve as a delivery device for drugs incorporated therein. The matrices described herein are formed from liquid precursor components which are able to form a three-dimensional network in the body at the site of need. The terms “matrix”, “sealant” and “three-dimensional network” are used synonymously herein. The terms “matrix” and “sealant” refer to the gelled formulation formed after the precursor solutions are mixed together and the crosslinking reaction has started in the gelled formulation.

Thus the terms “matrix” and “sealant” encompass partially or fully cross-linked polymeric networks. They may be in the form of a semi-solid, such as a paste, a solid a gel or a foam. Depending on the type of precursor materials, the matrix may be swollen with water but not dissolved in water, i.e. form a hydrogel which stays in the body for a certain period of time.

“Kit” as generally used herein refers to the precursor components needed to form a formulation, matrix or a foam in container.

“Fibrin formulation” as generally used herein refers to the fibrin precursor components, including fibrinogen and thrombin, in the period after mixing and before the crosslinking reaction of the monomeric fibrinogen molecules has started. The fibrin formulation is the state in which the precursor components, fibrinogen and thrombin, are mixed but the fibrinogen has not started to crosslink. With regard to the fibrinogen concentration, fibrin formulation refers to the mixing of the thrombin and fibrinogen precursor solution only, i.e. before optionally mixing a biocompatible gas. The fibrin formulation becomes a fibrin matrix or fibrin foam with crosslinking of the fibrinogen.

“Precursor composition” as generally used herein refers to the precursors needed to form a fibrin matrix, optionally a fibrin foam, before they are mixed together.

“Fibrin Matrix” as generally used herein means the product of a process in which the fibrin formulation, i.e. the fibrinogen of the fibrin formulation is crosslinked due to the interaction with a calcium source, thrombin and Factor XIIIa to form a three-dimensional network.

“Fibrin Foam” as generally used herein means the fibrin matrix which has a biocompatible gas incorporated therein in an effective amount to form a foam.

“Crosslinking” as generally used herein means the formation of covalent linkages.

“Supplemented matrix” as generally used herein refers to a matrix in which added bioactive factors are releasably incorporated therein.

“Controlled release” or “controlled delivery” as used herein have the same meaning and refer to retention of an bioactive factors in the fibrin matrix or fibrin foam. The terms “controlled release” or “controlled delivery” mean that both the amount of the agent released over time and/or the rate of release of the agent are controlled.

II. Fibrin Matrices, Fibrin Foams and Bioactive Factors

The fibrin formulations and matrices are prepared by combining a first solution, typically containing fibrinogen, and coagulation factor XIIIa, and a second solution, typically containing thrombin and calcium chloride in an aqueous base. In a preferred embodiment, the amount of thrombin is less than 0.3 U.I. thrombin per mg of fibrinogen. A fibrin foam is prepared by mixing a biocompatible gas to the fibrin formulation.

A. Fibrin Matrix or Foam

Fibrin is a natural material which has been reported for several biomedical applications. Fibrin matrices have been used as sealants due to their ability to bind to many tissues and their natural role in wound healing. Some specific applications include use as a sealant for vascular graft attachment, heart valve attachment, bone positioning in fractures and tendon repair. Additionally, these gels have been used as drug delivery devices, and for neuronal regeneration as well as material for cell in-growth matrices (U.S. Pat. No. 6,331,422 to Hubbell et al.).

The process by which fibrinogen is polymerized into fibrin has also been characterized. Initially, a protease cleaves the dimeric fibrinogen molecule at the two symmetric sites. There are several possible proteases than can cleave fibrinogen, including thrombin, peptidase, and protease III, and each one serves the protein at a different site. Once the fibrinogen is cleaved, a self-polymerization step occurs in which the fibrinogen monomers come together and form a non-covalently polymer gel held together by non covalent intermolecular forces. This self-assembly happens because binding sites become exposed after protease cleavage occurs. Once they are exposed, these binding sites in the centre of the molecule can bind to other sites on the fibrinogen chains, which are present at the ends of the peptide chains and lead to a gelation of the fibrin formulation Factor XIIIa, a transglutaminase, activated from factor XIII by thrombin proteolysis, may then covalently crosslink the fibrin formulation to a polymeric network. Other transglutaminases exist and may also be involved in covalent crosslinking and grafting to the fibrin network. Before the gelation state is reached other components like biocompatible gas, bioactive factors, granules or inert excipients can be added to the fibrin formulation.

Once a crosslinked fibrin matrix is formed, the subsequent degradation is tightly controlled. One of the key molecules in controlling the degradation of fibrin is α2-plasmin inhibitor. This molecule acts by crosslinking to α chain of fibrin through the action of factor XIIIa. By attaching itself to the matrix, a high concentration of inhibitor can be localized to the matrix. The inhibitor then acts by preventing the binding of plasminogen to fibrin and inactivating plasmin. The α2-plasmin inhibitor contains a glutamine substrate.

In another embodiment, the composition capable of forming a fibrin foam includes two precursor solutions and a biocompatible gas. Formation of fibrin foam is done by incorporating a biocompatible gas to the mixed precursor solutions, i.e., to the fibrin formulation before its gelation and crosslinking of the fibrin formulation. This could be done by the use of propellants as described in U.S. reissue Pat. No. RE39,321, the content of which is incorporated by reference. Or the incorporation of a biocompatible gas can be done by mechanically mixing the gas with the fibrin formulation. The biocompatible gas must be physiologically acceptable, suitable for pharmacological applications, and may include conventionally recognized gas, for example, CO₂, N₂, air or inert gas, such as chloroflurocarbons (CFC), for example, Freon® (DuPont), under pressure or not. Preferably, the biocompatible gas is air. In the alternative, the dry fibrin precursor components may be supplemented with material(s) which produce gas, and hence foaming, upon contact with the hydrating agent. In one preferred embodiment the volume of the fibrin formulation is about 80 to 120% of the volume of the biocompatible gas. Preferably volume of the fibrin formulation is about 100% of the volume of the biocompatible gas.

1. Fibrinogen

The first precursor solution contains fibrinogen, preferably in a concentration range between 10 to 130 mg fibrinogen per millilitre of precursor solution, more preferably between 30 to 60 mg fibrinogen per millilitre of precursor solution, even more preferably from between 20 to 58 mg fibrinogen per millilitre of precursor solution, and most preferably 30 to 55 mg fibrinogen per millilitre of precursor solution.

These concentration ranges of the precursor solutions generally results in fibrin formulations having a fibrinogen concentration of between 5 mg to 65 mg of fibrinogen per millilitre of fibrin formulation, preferably in between 7.5 mg to 30 mg fibrinogen per millilitre fibrin formulation, more preferably in between 10 to 58 mg fibrinogen per ml fibrin formulation and most preferably 15 mg to 27.5 mg fibrinogen per ml fibrin formulation. Fibrinogen is preferably solubilised in an aqueous buffer solution. Even more preferably, the fibrinogen dilution buffer comprises water, sodium citrate, preferably at a concentration of 25 mM, niacinamid, preferably at a concentration of 50 mM and histidine, preferably at a concentration of 100 mM, and has a preferably a pH of 7.3. It has been surprisingly found that fibrin formulations, matrices and foams having a low amount of fibrinogen still show excellent adhesive properties thereby reducing the risk of adverse reaction of the patient because the amount of non-autologous proteins is significantly lowered compared to commercially available fibrin sealants. A fibrin formulation with a fibrinogen concentration of 60 mg per ml of fibrin formulation and lower (but not lower than 7.5 mg fibrinogen per ml of fibrin formulation) shows , when transferred into a foam, very surprising healing results when treating chronic ulcers, in particular diabetic foot ulcers.

2. Thrombin

The concentrations of the fibrinogen solution and/or the thrombin solutions have a significant effect on the density of the resulting fibrin network and on the gelation and crosslinking speed of the final fibrin matrix or foam. Typically, the reduction of the amount of thrombin slows down the crosslinking process and contributes to form fibrin matrices or foams with a less dense network and longer manipulation time. Surprisingly, controlling the ratio of the amounts of thrombin with unchanged concentration of fibrinogen, leads to a more prolonged release of the growth factor, particularly, where a high concentration of growth factor is incorporated in the matrix or the foam. Furthermore, the ratio of the amount of thrombin to fibrinogen provides fibrin matrices or foams with a less dense network which is more suitable for cellular infiltration or in-growth and thus for wound healing.

In a preferred embodiment, the second precursor solution contains thrombin, wherein the thrombin amount is less than 0.3 U.I. thrombin per mg of fibrinogen, preferably in a range between 0.015 to 0.29 I.U. thrombin per mg of fibrinogen, more preferably in a range of 0.04 to 0.28 I.U. thrombin per mg of fibrinogen, and most preferably 0.08 I.U. thrombin per mg of fibrinogen. Thrombin is preferably solubilised in an aqueous buffer solution. Even more preferably, the thrombin dilution buffer comprises water, calcium chloride, preferably at a concentration of 40 mM, and sodium chloride, preferably at a concentration of 75 mM, and has preferably a pH of 7.3.

3. Calcium Source

A calcium ion source may be present in at least one of the precursor solutions and preferably in the second precursor solution. The calcium ion source is preferably CaCl₂*2H₂O, preferably in a concentration range between 1 to 10 mg per ml of precursor solution, even more preferably between 4 to 7 mg per ml of precursor solution, most preferably between 5 to 6 mg per ml of precursor solution.

4. Crosslinking Enzymes

An enzyme capable of catalysing the matrix formation after it has been activated, such as factor XIII, may be added to at least one of the precursor solution. Preferably, factor XIII is present in the fibrinogen precursor solution in a concentration range between 0.5 to 100 I.U. per millilitre of precursor solution, more preferably between 1 to 60 I.U. per millilitre of precursor solution, and most preferably between 1 to 10 I.U. per millilitre of precursor solution.

B. Bioactive Factors

Bioactive factors are releasably incorporated into the fibrin matrix Preferred bioactive factors are growth factors which are members of the transforming growth factor (TGF β) superfamily and members of the platelet derived growth factor (PDGF) superfamily. In particular, preferred members are PDGF, TGFβ, BMP, VEGF, and Insulin-like growth factor (IGF) and most preferred are PDGF AB, PDGF BB, PDGF D, TGFβ1, TGFβ3, VEGF 121 and IGF 1. These growth factors can be incorporated into the fibrin formulation and matrix during its formation and are incorporated either by electrostatic forces and/or chemical binding, like ionic, van-der-Waals forces or covalent binding. The added bioactive factors are present in the fibrin formulation, matrix or foam in a concentration range of between 1 to 20 μg per mg of fibrinogen, preferably from about 1.32 to 16 μg per mg of fibrinogen and most preferably from 4 to 12 μg per mg of fibrinogen. These concentration ranges are suitable for chronic skin wounds caused by diabetes, circulation problems or extended bed rests due to illness or operation (pressure sores).

Certain indications, like acute wounds, however, require much lower amounts of bioactive factor to acheive satisfying healing results. If growth factors, like PDGF are applied in too high amounts unwanted effects occur, like hypergranulation and necrosis of skin grafts. For these indications the bioactive factor is added in a concentration range of between about 0.0001 μg to about 1.5 μg bioactive factor per mg of fibrinogen. More preferred is a range of between about 0.0002 μg about 0.8 μg bioactive factor per mg of fibrinogen and most preferred a range of between about 0.0004 μg to about 0.5 μg bioactive factor per mg fibrinogen.

In another embodiment the growth factors are modified that it becomes capable of attaching to fibrin. This can be accomplished in several ways. By way of example, this may be achieved through the addition of a transglutaminase substrate domain to the growth factor or active fragment of the growth factor, resulting in a fusion protein having at least two domains, the growth factor or active fragment of the growth factor in one domain and the transglutaminase substrate domain as the second domain. Preferably the transglutaminase substrate domain is a factor XIII substrate domain. Optionally, the fusion protein may contain a degradation site.

In a preferred embodiment, the fusion protein comprises an amino acid sequence of SEQ ID NO:2 and SEQ ID NO:3 (referred herein as TG-PDGF).

Additional amino acid sequences may be added to the growth factor to include a degradation site and/or a substrate for a crosslinking enzyme (referred to hereinafter as the “TG-degr”-hook). The amino acid sequence is selected based on the structure of the growth factor. In case the growth factors are hetero- or homodimeric, the additional amino acids can be attached to the termini of either one or both of the chains. In the preferred embodiment, the TG-degr-sequence is attached to both chains. Depending on the structure of the growth factor, i.e., the location of the active centres within the protein, the TG-degr-sequence can be attached to the N and/or C-terminus of the chains. In a preferred embodiment, the TG-degr-sequence is attached to the N-terminus. When the growth factor is PDGF AB (heterodimeric) or TGFβ1 (homodimeric), the TG-degr-sequence is attached to the N-terminus of both chains.

The addition of a synthetic factor XIIIa substrate can be accomplished by expressing a fusion protein containing the native growth factor sequence and a factor XIIIa substrate at either the amino or carboxyl terminus of the fusion protein. This modification is done at the DNA level. Whole proteins present difficulty in that they are synthesized by solid phase chemical synthesis. The DNA sequence encoding the growth factor is adapted to optimal codon usage for bacterial expression. The DNA sequence is then determined for the desired Factor XIIIa substrate, using codons which occur frequently in bacterial DNA.

A series of gene fragments is designed prior to the DNA synthesis. Due to the error frequency of most DNA synthesis, which contains an error approximately every 50 bp, genes are constructed to be approximately 100 bp in length. This reduces the number of colonies that must be screened in order to find one containing the proper DNA sequence. The location at which one gene ends and the next begins is selected based on the natural occurrence of unique restriction enzyme cut sites within the gene, resulting in fragments (or oligonucleotides) of variable length. The process is greatly assisted by the use of software which identifies the location and frequency of restriction enzyme sites within a given DNA sequence.

Once the gene fragments have been successfully designed, common restriction enzyme sites are included on the ends of each fragment to allow ligation of each fragment into a cloning plasmid. For example, adding EcoRI and HindIII sites to each gene fragment allows it to be inserted into the polylinker cloning region of pUC 19. The 3′ and 5′ single strands of each gene fragment are then synthesized using standard solid phase synthesis with the proper sticky ends for insertion into the cloning vector. Following cleavage and desalting, the single stranded fragments are then purified by PAGE and annealed. After phosphorylation, the annealed fragments are ligated into a cloning vector, such as pUC 19.

Alternatively, two DNA molecules can be spliced together using overlap extension PCR (Mergulhao et al. Mol Biotechnol., 12(3):285-7 (1999)). First, genes are amplified by means of polymerase chain reactions (PCR) carried out on each molecule using oligonucleotide primers designed so that the ends of the resultant PCR products contain complementary sequences. When the two PCR products are mixed, denatured and reannealed, the single-stranded DNA strands having the complementary sequences anneal and then act as primers for each other. Extension of the annealed area by DNA polymerase produces a double-stranded DNA molecule in which the original molecules are spliced together. Gene splicing by overlap extension (SOE), provides for recombining DNA molecules at precise junctions irrespective of nucleotide sequences at the recombination site and without the use of restriction endonucleases or ligase. The SOE approach is a fast, simple, and extremely powerful, way of recombining and modifying nucleotide sequences.

Following ligation, the plasmids are transformed into DH5-F′ competent cells and plated on Isopropyl-D-Thiogalactopyranoside (IPTG)/Bromo-4-chloro-3-indolyl-D-Galactopyranoside (X-gal) plates to screen for insertion of the gene fragments. The resulting colonies which contain gene fragment are then screened for insertion of the proper length. This is accomplished by purifying plasmid from colonies of transformed cells by alkaline lysis miniprep protocol and digesting the plasmid with the restriction enzyme sites present at either end of the gene fragment. Upon detection of the fragments of the proper length by agarose gel electrophoresis, the plasmids are sequenced.

When a plasmid containing a gene fragment with the proper sequence is identified, the fragment is then cut out and used to assemble the full gene,

Each time one plasmid is cut with the enzymes at the insertion points and purified from an agarose gel after dephosphorylation of the plasmid. Meanwhile, a second plasmid containing the fragment to be inserted is also cut and the fragment to be inserted is purified from an agarose gel. The insert DNA is then ligated into the dephosphorylated plasmid, This process is continued until the full gene is assembled. The gene is then moved into an expression vector, such as pET 14b and transformed into bacteria for expression. After this final ligation, the full gene is sequenced to confirm that it is correct.

Expression of the fusion protein is accomplished by growing the bacteria until they reach mid-log phase growth and then inducing expression of the fusion protein. Expression is continued for approximately 3 hours and the cells are then harvested. After obtaining a bacterial cell pellet, the cells are lysed. The cell membranes and debris are removed by washing the cell lysate pellet with Triton X100, leaving the inclusion bodies in relatively pure form. The fusion protein is solubilized using high urea concentrations and purified by histidine affinity chromatography. The resulting protein is then renatured gradually by dialysis against a slowly decreasing amount of urea and lyophilized.

III. Methods for Incorporation and/or Release of Fusion Proteins

The disclosed fusion protein supplemented fibrin matrices or foams are formed by crosslinking of the fibrinogen monomers. A calcium source, thrombin, fibrinogen and at least one fusion protein form the supplemented fibrin matrix. In another embodiment, a calcium source, thrombin, fibrinogen, at least one fusion protein and a biocompatible gas form the supplemented fibrin foam

Exogenous peptides can be designed as fusion proteins which include two domains, where the first domain is a bioactive factor, such as a peptide, protein, or polysaccharide, and the second domain is a substrate for a cross-linking enzyme, such as Factor XIIIa. Factor XIIIa is a transglutaminase that is active during coagulation. This enzyme, formed naturally from factor XIII by cleavage by thrombin, functions to attach fibrin chains to each other via amide linkages, formed between glutamine side chains and lysine side chains. Factor XIIIa also attaches other proteins to fibrin during coagulation, such as the protein alpha 2 plasmin inhibitor. The N-terminal domain of this protein, specifically the sequence NQEQVSP (SEQ ID NO:1), has been demonstrated to function as an effective substrate for factor XIIIa. A second domain of this peptide can contain a bioactive factor, such as a peptide, protein, or a polysaccharide (Sakiyama-Elbert, et al, J. Controlled Release, 65:389-402 (2000)). Such fusion proteins may be used to incorporate bioactive factors (e.g. growth factors) within fibrin during coagulation via a factor XIIIa substrate.

Surprisingly, reducing the amount of thrombin (keeping the amount of fibrinogen constant) allows for prolonged controlled release of the fusion protein from the fibrin matrix or foam. Reducing the amount of thrombin allows for a control on the amount of growth incorporated and thus released over time and a control of the rate of release of the growth factor. This effect is independent to the amount of growth factor initially incorporated in the fibrin matrix or foam. In one preferred embodiment, thrombin is used in an amount of less than 0.3 I.U. thrombin per mg of fibrinogen, preferably in a range of between 0.015 to 0.29 I.U. thrombin per mg of fibrinogen, more preferably in a range between 0.04 to 0.28 I.U. thrombin per mg of fibrinogen, most preferably between 0.06 to 0.1 I.U. thrombin per mg of fibrinogen, and in particular applications around 0.08 I.U. thrombin per mg of fibrinogen.

In a general method for preparing a fibrin matrix comprising at least one fusion protein covalently linked onto it, the method includes the steps of:
(i) providing a fibrinogen solution;
(ii) providing a thrombin solution wherein the amount of thrombin is less than 0.3 I.U. thrombin per mg of fibrinogen;
(iii) providing at least one fusion protein comprising a first domain comprising a bioactive factor and a second domain comprising a transglutaminase substrate domain; and
(iv) mixing components provided in steps (i), (ii) and (iii) to crosslink the matrix material such that the fusion protein is covalently linked to the matrix through the second domain.

The matrix can be in a form of a foam which requires adding the biocompatible gas into the fibrin formulation.

In a general method for preparing a fibrin foam comprising at least one fusion protein covalently linked onto it, the method includes the steps of
(i) providing a fibrinogen solution;
(ii) providing a thrombin solution wherein the amount of thrombin is less than 0.3 I.U. thrombin per mg of fibrinogen;
(iii) providing at least one fusion protein comprising a first domain comprising a platelet derived growth factor (PDGF) and a second domain comprising a transglutaminase substrate domain;
(iv) providing a biocompatible gas; and
(v) mixing components provided in steps (i), (ii), (iii) and (iv) to form a fibrin formulation.

The controlled delivery fibrin matrix or foam obtained are characterized in that no more than 25% of growth factor is released after incubation of the controlled delivery fibrin foam during 3 days at 37° C. in a buffer solution.

In one embodiment, the fusion protein amount is in range from about 1 to 20 μg/mg of fibrinogen, preferably from about 1.32 to 16 μg/mg of fibrinogen, even more preferably from about 4 to 12 μg/mg of fibrinogen. In another embodiment the added bioactive factor is in a range of between 1.5 μg to 0.0001 μg bioactive factor per mg of fibrinogen. More preferred is a range of between 0.8 μg to 0.0002 μg bioactive factor per mg of fibrinogen and most preferred a range of between 0.5 μg to 0.0004 μg bioactive factor per mg fibrinogen

In a preferred embodiment, the fibrin formulation with or without containing a biocompatible gas is applied to the site of need in or on the body and crosslink in situ in or on the body. The fibrinogen and thrombin precursor solutions should be separated prior to application of the mixture, i.e., the fibrin formulation, to the body to prevent combination or contact with each other under conditions that allow polymerization of the solutions. To prevent contact prior to administration, a kit which separates the solutions from each other may be used. Upon mixing under conditions that allow polymerization, the compositions form a fibrin matrix or foam which optionally can be supplemented with a bioactive factor. Depending on the precursor solutions and their concentrations, gelation can occur quasi-instantaneously after mixing. Such a fast gelation, makes the application or injection, i.e. squeezing of the gelled or foamed material through the injection needle, almost impossible.

Surprisingly, amounts of thrombin and fibrinogen such that the amount of thrombin is less than 0.3 I.U. thrombin per mg of fibrinogen are suitable for forming a fibrin foam by mixing a biocompatible gas into the fibrin formulation which can optionally supplemented with bioactive factors, preferably fusion proteins. Upon mixing of the precursor solutions gelation occurs fast enough to produce a foam but is slow enough for allowing the foam to be applied or injected at the site of need before its full gelation and consecutive clogging of the application or injection device. The foam sticks to the surface where it is applied. Thus by converting the fibrin formulation into a foam, the fibrin formulation does not run off the surface where it is applied (which would occur with a non foamed fibrin formulation). This method and the ratio of thrombin and fibrinogen are well suited to apply or inject the material in less than 1 minute from the mixing of the precursor solutions, preferably in less than 30 seconds and more preferably within 15 seconds. The applied or injected fibrin foam is adhesive enough to stay at the administration site and is malleable enough to be administered with the desired shape.

In one embodiment, fibrinogen, which may also contain aprotinin to increase stability, is dissolved in a buffer solution at physiological pH, ranging from pH 6.5 to 8.0, preferably ranging from pH 7.0 to 7.5. The buffer solution for the fibrinogen can comprises water, sodium citrate, preferably at a concentration of 25 mM, niacinamid, preferably at a concentration of 50 mM and histidine, preferably at a concentration of 100 mM, and has a preferably a pH of 7.3. Thrombin in a calcium chloride buffer (e.g. concentration range of from 40 to 50 mM) is prepared. The fibrinogen is then stored separately from the thrombin solution. The fibrinogen and the thrombin solutions can be stored frozen to enhance storage stability or one or the other can be lyophilized. Prior to use the fibrinogen solution and the thrombin solution are defrosted (when necessary) or reconstituted in buffer solution and mixed. In another embodiment, fibrinogen and thrombin can be stored separately from the calcium source. In still another embodiment, the fibrinogen can be stored with the calcium source and separated from the thrombin.

### IV. Kits

In another embodiment, a kit, which contains fibrinogen, thrombin, and optionally (i) bioactive factors, (ii) calcium source and (iii) a biocompatible gas, is provided. Optionally, the kit may also contain a crosslinking enzyme, such as Factor XIIIa. Preferred bioactive factors are growth factors which are members of the transforming growth factor (TGF β) superfamily and members of the platelet derived growth factor (PDGF) superfamily. In particular, preferred members are PDGF, PDGF A, PDGF B, PFGF D, PDGF BB, PDGF AB, TGFβ, BMP, VEGF, and Insulin-like growth factor (IGF) and most preferred are PDGF AB, TGFβ1, TGFβ3, BMP2, BMP7, VEGF 121 and IGF 1. Bioactive factors can also be in the form of fusion protein which contains a bioactive factor, a substrate domain for a crosslinking enzyme and optionally a degradation site between the substrate domain and bioactive factor. The fusion protein may be present in either the fibrinogen or the thrombin solution. In a preferred embodiment the fibrinogen solution contains the fusion protein. The biocompatible gas may be present in either the fibrinogen solution or the thrombin solution or in a separate container. The precursor solutions are preferably mixed by a two way syringe device, in which mixing occurs by squeezing the contents of both syringes through a mixing chamber and/or needle and/or static mixer. The mixed precursor components, i.e. the fibrin formulation, can be sprayed or brushed or applied by needles to the site of need. Alternatively the precursor components can be mixed by syringe to syringe mixing, i.e. by connecting two containers containing the precursor solutions and pushing the contents from one container to the other thereby mixing the solutions. If a biocompatible gas is added it can be added to the already mixed precursor components, i.e. the fibrin formulation or it can be added at the same time the two precursor solutions are mixed.

In a preferred embodiment both fibrinogen and thrombin are stored separately in lyophilised form. Either of the two can contain the bioactive factor. Prior to use, the fibrinogen dilution buffer is added to the lyophilized fibrinogen, the buffer may additionally contain aprotinin. The lyophilized thrombin is dissolved in the calcium chloride solution.

The fibrinogen and the thrombin solutions are contained or placed in separate containers/vials/syringe bodies and mixed by a two way connecting device, such as a two-way syringe. Optionally, the containers/vials/syringe bodies are bipartite thus having two chambers separated by an adjustable partition which is perpendicular to the syringe body wall. One of the chambers contains the lyophilised fibrinogen or thrombin, while the other chamber contains an appropriate buffer solution. When the plunger is pressed down, the partition moves and releases the buffer into the fibrinogen chamber to dissolve the fibrinogen. In order to form a fibrin foam, a biocompatible gas can be added to any of the containers/vials/syringe bodies containing the fibrinogen solutions or the thrombin solutions or can be stored in a separate container and added at any time to the precursor solutions or fibrin formulation before it is completely gelled Once both fibrinogen and thrombin are dissolved, both bipartite syringe bodies are attached to a two way connecting device and the contents are mixed by squeezing them through the injection needle attached to the connecting device. Optionally, the connecting device contains a static mixer to improve mixing of the contents.

In a preferred embodiment the volume of the biocompatible gas is about 80 to 120% of the volume of the fibrin formulation, preferably 100%. This ratio results in window of approximately 15 seconds during which the foaming process has started and produces a surface adhesive material that can be applied or injected at the site of need before full crosslinking has occurred. This allows applying the material to a surface which is not horizontal and preventing the material to run off the surface. This is particularly useful for wound healing indication where the surface to be treated is not horizontal such as the feet or legs of a patient.

### V. Methods of Use

The disclosed fibrin formulation, fibrin matrix or fibrin foam can be used for repair, regeneration, or remodelling of tissues, and/or release of bioactive factors, once placed in the body. For most healing applications it is favourable to add the appropriate bioactive factors, however certain indications show healing results in particular just with the fibrin matrix and in particular the before described fibrin foam.

The controlled delivery matrices or foams of the present invention can be used in the treatment of acute and/or chronic wounds, preferably wherein the wound is a chronic ulcer or the wound is an acute wound. Acute wounds include but are not limited to wounds which require skin grafting procedures. This includes skin areas which are wounded due to burns which are subsequently covered by autologous skin parts harvested from other areas of the body. The skin grafts get meshed and put on the wounded area in a stretched manner to cover as much as the wound area as possible. The fibrin formulations and matrix of the present invention will be applied to the wounded area and keep the skin graft in place due to its adhesive properties. The fibrin formulation can be applied as an adjunct to other fixation means like staples or can also be used on its own also dependent on the size and location of the wound and preference of the surgeon. In addition to its adhesive properties, the fibrin matrices of the present invention diminishes the risk of dislocation of the graft and enhances the wound healing, i.e., the growing together of the skin graft to the underlying tissue surface and also enhances the skin growth in the meshes of the graft to achieve a faster and cosmetically more acceptable result than the current treatments on the market. Another example of acute wounds are wounds created in the inner of the body by procedures which are summarized as flap surgery. These include all kinds of plastic surgery, like face lift, in which certain parts or layers of the patient's body are separated, manipulated and then reattached to the undenying tissue. The fibrin formulations, matrices and foams of the present invention show through their adhesive and healing properties advantageous effects when applied as a layer between the separated flap and the underlying tissue. By applying the fibrin matrix of the present invention complications during the healing process like seroma formation are substantially decrease, dislocation of the flap is diminished and the healing time is In manner cases faster than without applying the matrices and foams of the present invention.

The fibrin matrices and in particular the fibrin foams of the present invention reduces the size of chronic wounds, in particular when these chronic wounds are caused by diabetes or circulation problems as the underlying cause of, and they support the wound closure as well. Fibrin foams have the advantage that they stick to wounds even when the wound is located in non-horizontal positioned wounds without running off the wound site. The fibrin foams show beneficial effects also without the addition of bioactive factors.

Cells can also be added to the matrix prior to or at the time of implantation, or even subsequent to implantation, either at or subsequent to cross-linking of the polymer to form the matrix. This may be in addition to or in place of crosslinking the matrix to produce interstitial spacing designed to promote cell proliferation or in-growth.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Formation of TG-PDGF.AB

PDGF AB used in these experiments consisted of a PDGF A chain of 110 amino acids and a PDGF B chain of 109 amino acids. This form of PDGF AB can be found naturally in the human body.

The PDGF AB sequence was modified to allow for covalent binding to a fibrin matrix. Additional 21 amino acids, the TG-hook containing a plasmin degradation site, were attached to both of the N termini of the PDGF AB, as follows:

TG-N_{(A)} . . . C_{(A)}

C_{(B)} . . . N_{(B)}-TG

N refers to the N-terminus; C refers to the C-terminus; (A) refers to the A-chain; and (B) refers to the B-chain.

The amino acid sequence of TG-PDGF A is:

(SEQ ID NO: 2)
MNQEQVSPLPVELPLIKMKPHSIEEAVPAVCKTRTVI-
YEIPRSQVDPTSANFLIWPPCVEV-
KRCTGCCNTSSVKCQPSRVHHRSVKVAKVEYVRK-
KPKLKEVQVRLEEHLECACATTSLNPDYREEDTDVR.

The amino acid sequence of TG-PDGF B is:

(SEQ ID NO: 3)
MNQEQVSPLPVELPLIKMKPHSLGSLTIAEPAMIAECK-
TRTEVFEISRRLIDRTNANFLVWPPCVEVQRCSGCCNNRNVQCRPTQ
VQLRPVQVRKIEIVRKKPIFKKATVTLEDHLACKCETVAAARPVT.

The A chain and the B chain of the heterodimer TG-PDGF AB were expressed separately in a bacterial system. The inclusion bodies of the bacteria cells were solubilized to release the A or the B chain, respectively. Both, the A and B chain solution were purified (separately) by using a cationic exchange column. Subsequently the A and the B chain were reduced/denaturized and precipitated. The precipitates were dissolved and the A and the B chain solution were mixed for the refolding step. The refolding to TG-PDGF AB occurred in a buffer solution over a period of three to five days. The refolded protein was purified by a two-step purification process, which contained a cationic exchange column followed by a gel filtration column.

### Release Study Protocol

For each experiment, 100 μL-fibrin formulation gels were made in triplicates using the DuPlojectTM devices from Baxter. These 2-syringe devices allow mixing of equal amounts of the fibrinogen precursor solution containing TG-PDGF.AB and the thrombin precursor solution contained in the two syringes, i.e., each in one compartment of the two syringe device. The first, fibrinogen precursor solution contained 50 mg fibrinogen/ml of fibrinogen precursor solution (equivalent to 25 mg fibrinogen/ml fibrin formulation) in in buffer solution containing water, sodium citrate 25 mM, niacinamid 50 mM and histidine 100 mM. The first precursor solution has a pH of 7.3. TG-PDGF.AB is added to the fibrinogen precursor solution at concentration of 66, 200 or 600 μg/ml of the precursor solution, which is equivalent to 1.32, 4 and 12 μg TG-PDGF AB/mg of fibrinogen. The second precursor solution can be contained in a syringe which contains thrombin at concentrations of 4, 15, 31, 62, 125 and 250 I.U. thrombin/ml thrombin precursor solution which is equivalent to 0.08, 0.3, 0.62, 1.24, 2.5, 5 I.U thrombin/mg of fibrinogen diluted in a buffer containing calcium chloride 40 mM and sodium chloride 75 mM. The syringe solutions are mixed in equal volumes.

The fibrin formulation gels were left drying at 37° C. for one hour. They were inserted in 15 ml-falcon tubes containing 10 ml release buffer (TRIS 10 mM, NaCl 70 mM, KCl 1.3 mM, BSA 0.1%, pH 7.4) and incubated for 72 hours in an incubator at 37° C. 100 μL release buffer aliquots were taken at appropriate time points (approximately 6, 24, 48 and 72 hours). PDGF-AB concentrations contained in the release buffer at different time points were determined using an in-house ELISA assay.

### Example 1

### Release Rates of Two Different Concentrations of TG-PDGF.AB from a Fibrin Matrix

A release study using the fibrin formulations as described in the release study protocol was done 5 times (with the same or different lots, on different days) with fibrinogen solution containing either 66 or 600 μg TG-PDGF.AB/ml fibrinogen precursor solution. An average release rate was calculated using these 5 experiments.

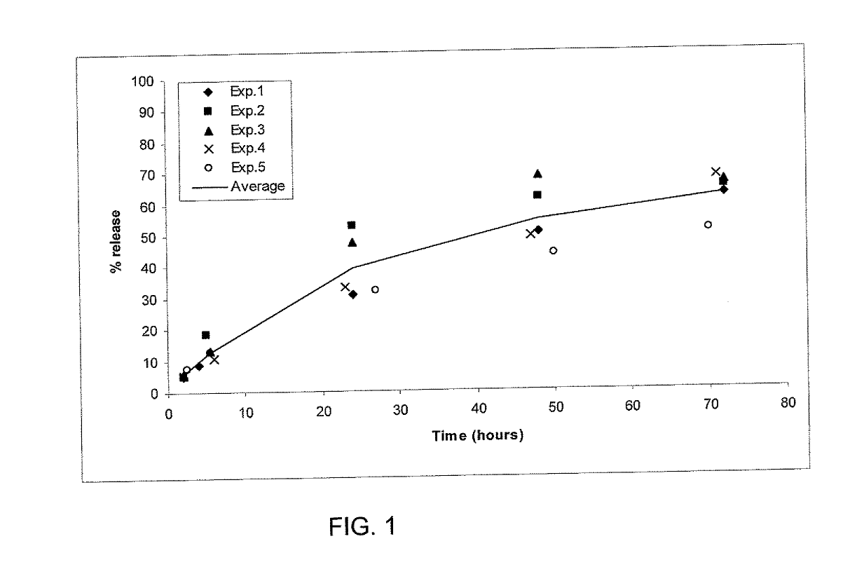

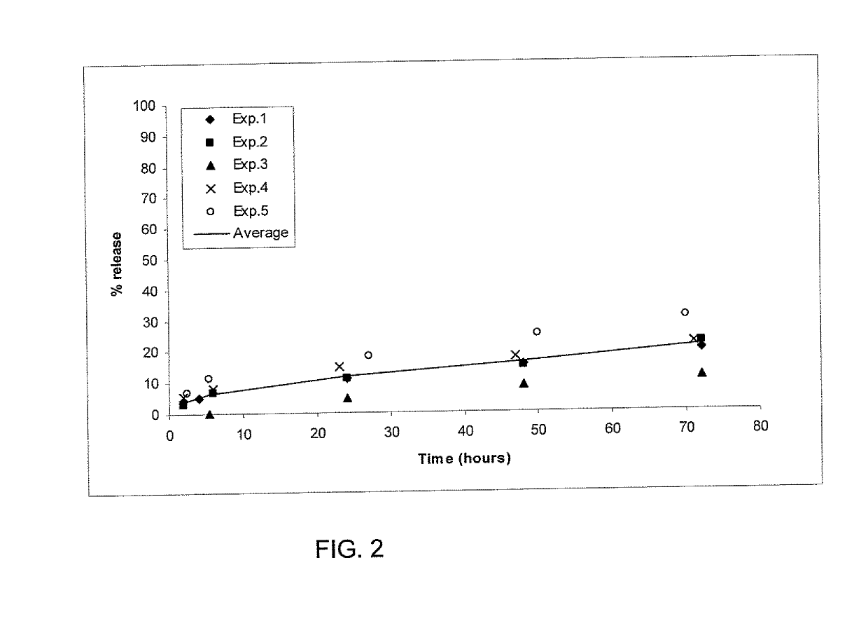

The release rate of the higher dose (600 μg TG-PDGF.AB/ml fibrinogen precursor solution) (FIG. 1) is higher than the release rate of the lower dose (66 μg TG-PDGF.AB/ml fibrinogen precursor solution) (FIG. 2) over 70 hours: 62% and 21% respectively of the initial PDGF amount is released within 70 hours, which indicates that the chemical binding is not as efficient with higher concentrations of TGPDGF than with lower concentrations.

### Example 2

### Influence of Thrombin Concentration on the Release of TG-PDGF.AB

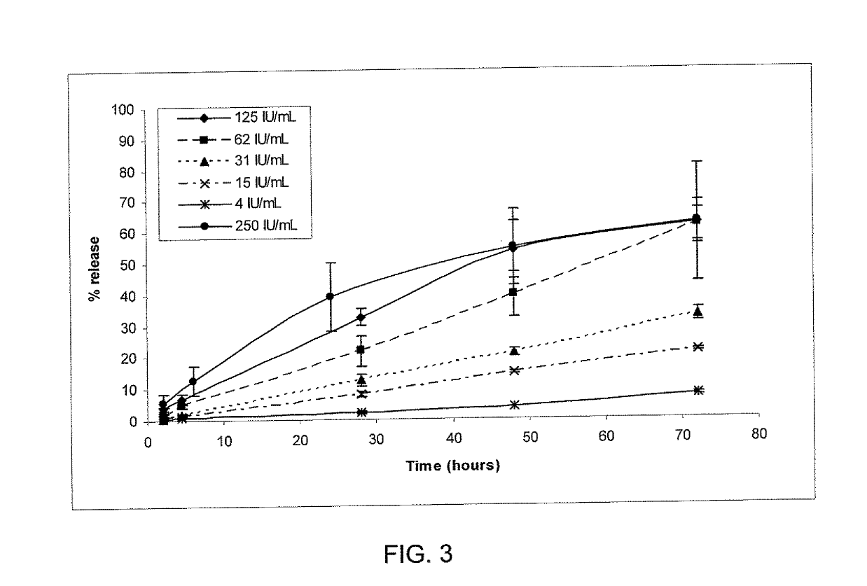

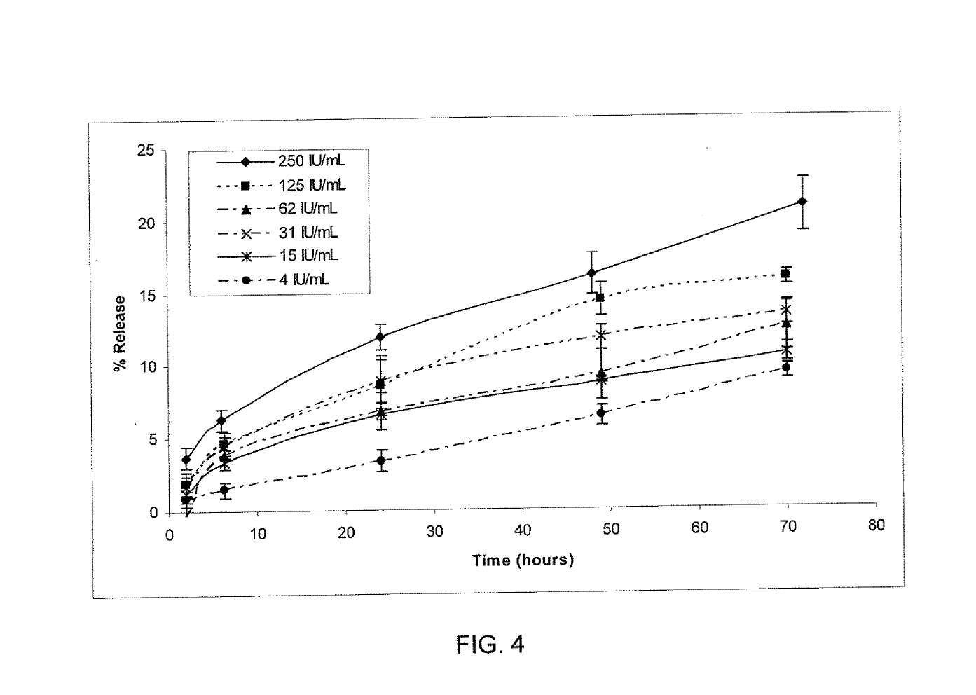

A release study was performed using different amounts of thrombin (4, 15, 31, 62, 125 and 250 IU thrombin/mi thrombin precursor solution (equivalent to 0.08, 0.3, 0.62, 2.5 and 5 I.U. thrombin/mg fibrinogen)), the fibrinogen solution remaining unchanged (50 mg fibrinogen/ml fibrinogen precursor solution (equivalent to 25 mg fibrinogen/ml fibrin formulation) and 600 μg TG-PDGF.AB/ml fibrinogen precursor solution (12 μg TG-PDGF.AB/mg fibrinogen (see FIGS. 3) and 66 μg TG-PDGF.AB/ml fibrinogen precursor solution (equivalent to 1.32 μg TG-PDGF.AB/ml fibrinogen (FIG. 4).

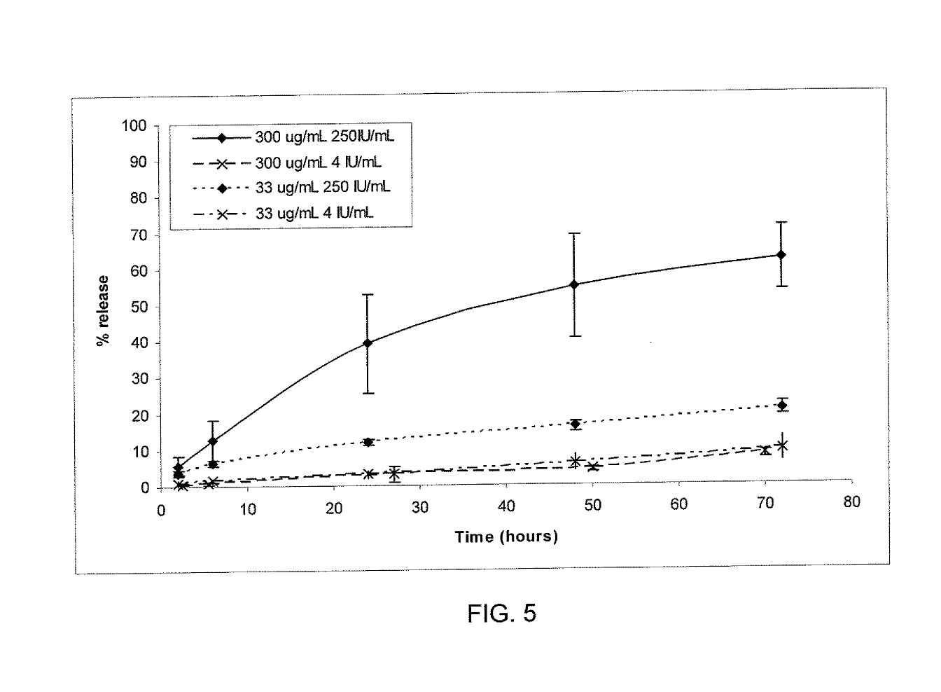

Data corresponding to 250 IU thrombin/ml thrombin precursor solution and 4 IU thrombin/ml thrombin precursor solution of FIGS. 3 and 4 are presented are shown in FIG. 5. For both doses, decreasing the thrombin concentration leads to a lower release rate.

### Example 3

### Release Study with Differing Amounts of Factor XIII

A release study was performed adding different amounts of factor XIII in the fibrinogen solution (0, 0.2, 2 and 20 I.U. factor XIII/ml fibrinogen precursor solution i.e. 0, 0.1, 1 and 10 I.U. factor XIII/ml fibrin formulation). The fibrinogen concentration for all samples was 50 mg fibrinogen/ml of fibrinogen precursor solution.

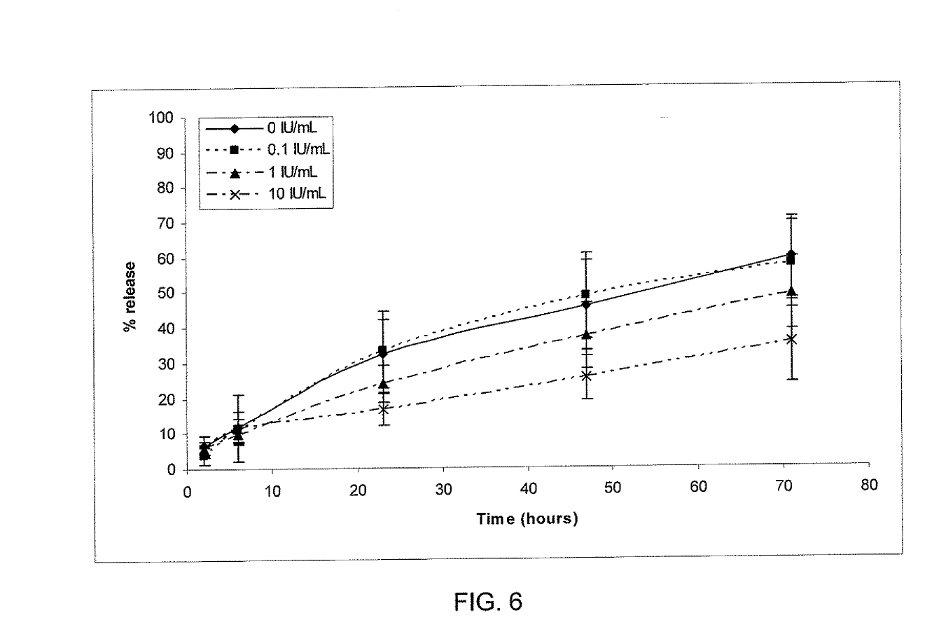

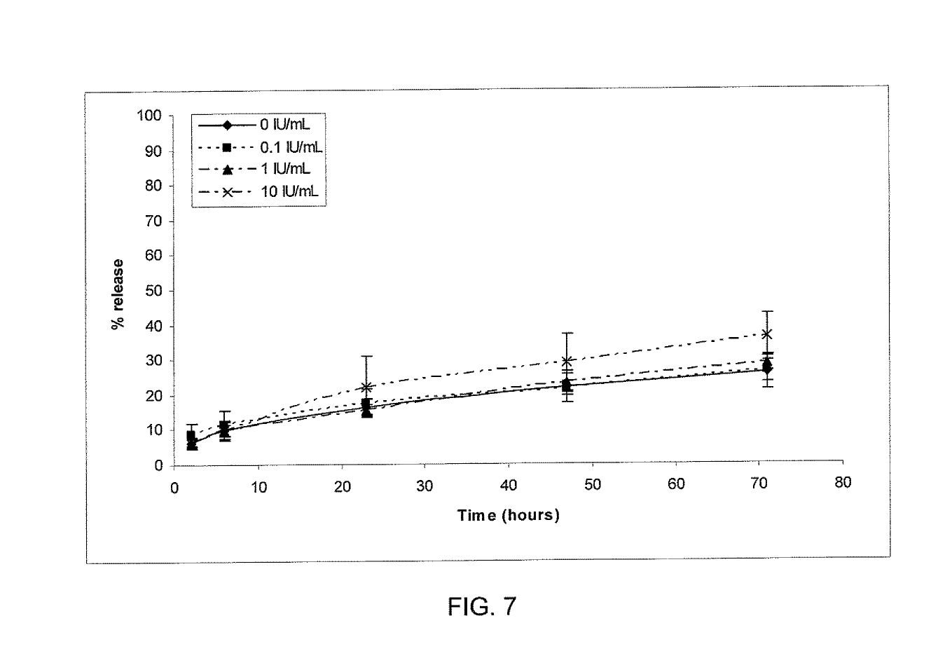

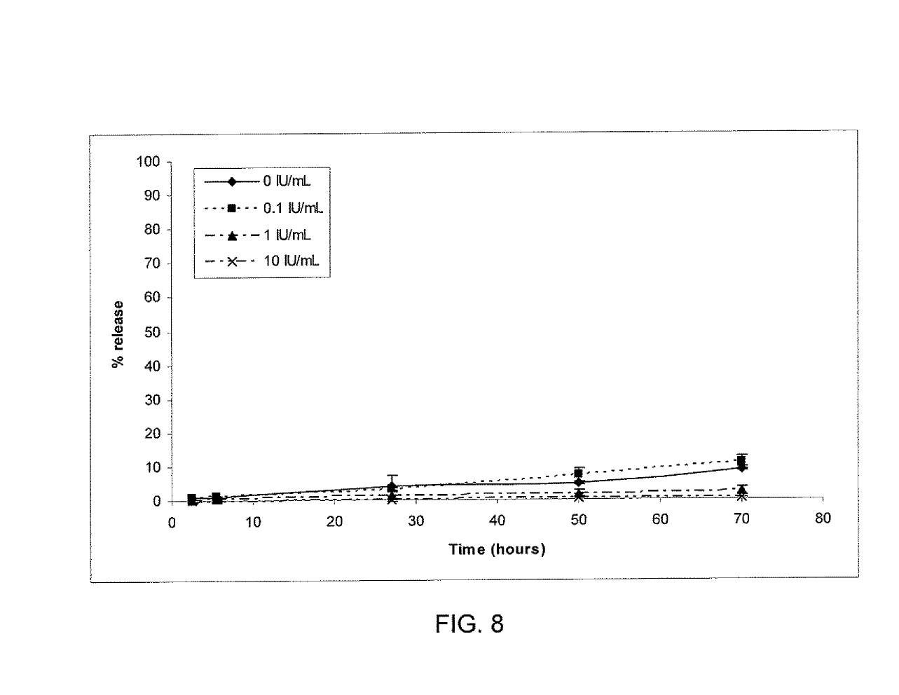

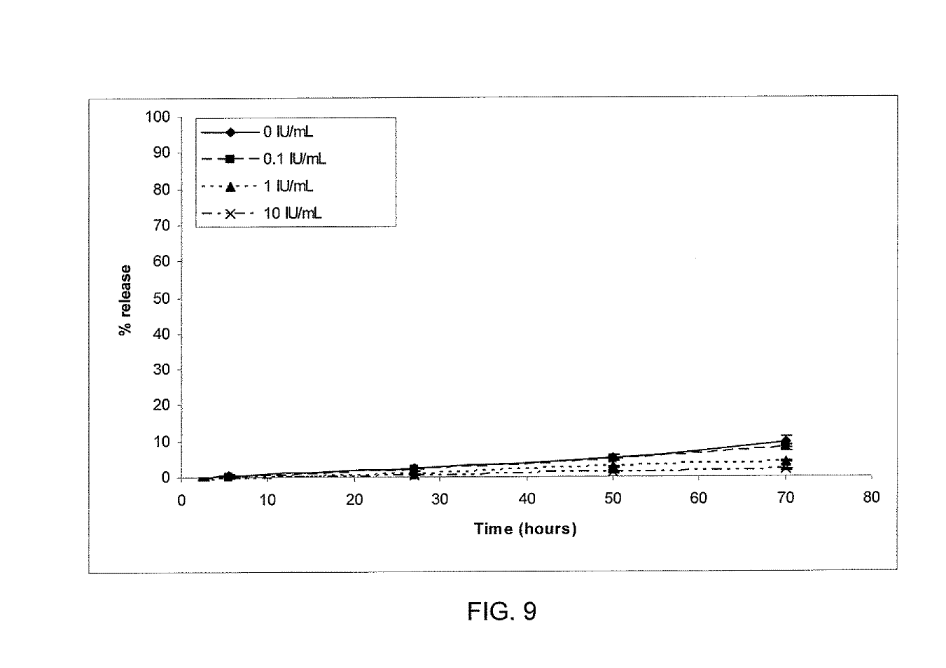

This experiment was done for the higher (300 μg TG-PDGF.AB/ml fibrin formulation (equivalent to 6 μg TG-PDGF.AB/mg fibrinogen) and lower concentrations (33 μg TG-PDGF.AB/ml fibrin formulation (equivalent to 0.66 μg TG-PDGF.AB/mg fibrinogen) using 250 IU thrombin/ml thrombin precursor solution (equivalent to 5 I.U. thrombin/mg fibrinogen) (FIGS. 6 and 7 respectively), and for the higher and lower doses using 4 IU thrombin/ml thrombin precursor solution (equivalent to 0.08 I.U. thrombin/mg fibrinogen) (FIGS. 8 and 9 respectively).

For 250 IU thrombin/ml thrombin precursor solution, increasing factor XIII concentration leads to a lower release for the higher concentration of TG-PDGF.AB (60% to 35% release). This has no significant influence on the release rate for the lower concentration of TG-PDGF.AB.

For 4 IU thrombin/ml thrombin precursor solution, increasing factor XIII concentration has no influence on the release rate for both concentrations of TG-PDGF.AB as the release rate is already low (around 10%).

### Example 4

### Release Study from Fibrin Foam

### Material and Methods

The test items, fibrin foams, were prepared by mixing the content of two syringes through a mixer three times back and forth. The first syringe contained 0.5 ml of 50 mg fibrinogen/ml fibrinogen precursor solution (equivalent to 25 mg fibrinogen/ml fibrin formulation), the second contained 0.5 ml of 4 IU thrombin/ml thrombin precursor solution (equivalent to 0.08 I.U. thrombin/mg fibrinogen). The fibrinogen precursor solution contained 66 μg/ml fibrinogen per ml (equivalent to 1.32 μg/mg fibrinogen. The thrombin precursor solution contained additionally 1 ml of air. The fibrinogen concentration for all samples was 50 mg fibrinogen/ml of fibrinogen precursor solution.

6 samples (samples with buffer changed) or 4 replicates (sample with buffer not changed) of each test item were prepared. The test items were prepared in 2.5 ml syringes from which the ends had been cut, used as moulds. Samples were dried for 1 hour at 37° C. and weighed before being assayed in the buffer in order to estimate the total amount of TG-PDGF.AB contained in the initial test items.

Samples with buffer changed: the test items were incubated in 10 ml release buffer in 15 ml falcon tubes and 100 μL aliquots of this buffer were taken at each time point and frozen at −20° C. until further analysis. At each time-point (twice a day) and until complete degradation of the samples occurred, the buffer was removed and 10 ml fresh release buffer added to the samples.

Samples with buffer not changed: the test items were incubated in 10 ml release buffer in 15 ml falcon tubes and 500 μL aliquots of this buffer were taken at each time point and frozen at −20° C. until further analysis. The buffer was not changed at each time point.

An ELISA system was used to quantify PDGF AB (both TG-PDGF.AB and PDGF-AB after cleaving the TG sequence; ELISA does not differentiate) contained in the buffer aliquots taken at the various time points. The PDGF-AB concentrations of the release samples were calculated from the Optical Density values obtained by ELISA, with all calculations performed and graphs plotted using Microsoft EXCEL.

### Results

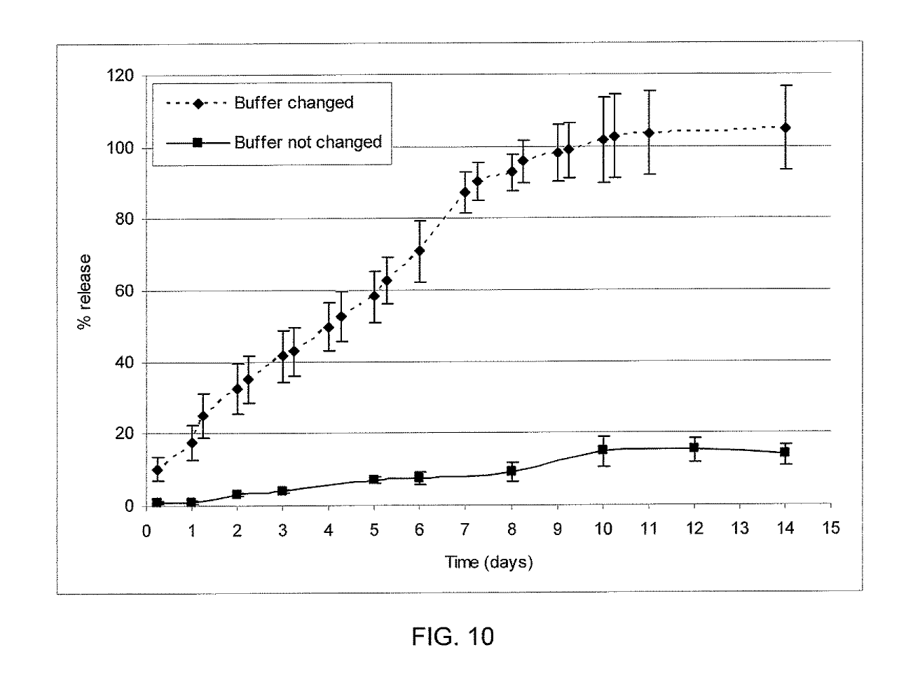

When the buffer was changed, the test items degraded (after 14 days, all samples had disappeared). On the opposite, if the buffer was not changed, the test items were intact (as assessed visually) after 14 days incubation in buffer. The percentage of PDGF-AB released from each test items was calculated and plotted against time (see FIG. 10). The results show that 100% of TG-PDGF.AB initially incorporated in the test items were recovered upon degradation of the test items, whereas only 14% were released when buffer was not changed the remaining quantities remained in the foam.

### Example 5

### Comparison of TG-PDGF.AB and Native PDGF-AB Release from Fibrin Foam

Fibrin foam samples were prepared as described in example 4 with 50% air of the total volume. The samples were weighed so as to determine the total amount of fibrin/TG-PDGF.AB contained in the fibrin foam clots (corresponding to 100% level on the graph). The fibrin foam clots were prepared in triplicates. Three TG-PDGF.AB and PDGF AB concentrations were tested: 66 (low), 200 (middle) and 600 (high) μg/ml of fibrinogen precursor solution. These concentrations correspond to 16.5, 50 and 150 μg/ml in the fibrin foam or 1.32, 4, 12 μg TG-PDGF.AB/mg of fibrinogen or FDGF AB7 mg of fibrinogen

After preparation, the fibrin foam samples were incubated for 3 days at 37° C. in release buffer, and aliquots were taken at 4 time points: 6 h, 25 h, 48 h and 75 h. The concentration of PDGF contained in the release buffer at these time points was determined by ELISA.

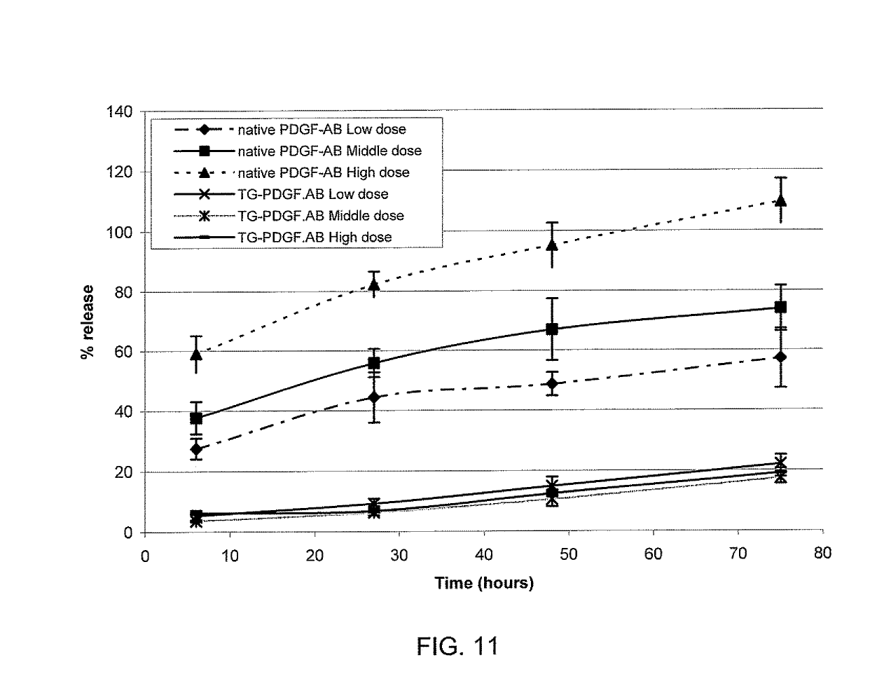

FIG. 11 shows the release profiles of TG-FDGF.AB and native PDGF-AB from fibrin foam clots for all three concentrations.

The release rates of TG-PDGF.AB versus native PDGF.AB were 22% vs. 57%, 17% vs. 74% and 19% vs. 110% for low, middle and high concentrations. Although the retention of TG PDGF. AB in the fibrin foam were higher compared to native PDGF AB over 70 hours the results show that a significant percentage of the low and middle concentrations of native PDGF AB still were retained in the fibrin foam after 70 hours. Native PDGF AB seems to be retained better the lower the concentration of PDGF in the fibrin foam is.

### Example 6

### Release Study from Diluted Fibrin and Low TG-PDGF.AB Concentration

### Material and Methods

A fibrin formulation was prepared from mixing 1 ml of a fibrinogen precursor solution containing TG-PDGF,AB with 1 ml of a thrombin precursor solution (each precursor solution in a syringe) using the Baxa Red Connector. Thorough mixing was achieved by transferring the content of syringes back and forth five times. 100 μl fibrin formulations of the following compositions were made in triplicates:
Fibrin formulation 1: 76.5 mg fibrinogen/ml fibrinogen precursor solution (equivalent to 38.25 mg fibrinogen/ml fibrin formulation), 5.1 I.U. thrombin/ml thrombin precursor solution (equivalent to 0.067 I.U. thrombin/mg fibrinogen) and 1 μg TG-PDGF.AB/ml fibrin formulation (equivalent to 0.013 μg TG-PDGF.AB/mg fibrinogen).
Fibrin formulation 2: 76.5 mg fibrinogen/ml fibrinogen precursor solution (equivalent to 38.25 mg fibrinogen/ml fibrin formulation), 5.1 I.U. thrombin/ml thrombin precursor solution (equivalent to 0.067 I.U. thrombin/mg fibrinogen) and 11 μg TG-PDGF.AB/ml fibrin formulation (equivalent to 0.144 μg/mg fibrinogen).
Fibrin Formulation 3: 38.2 mg fibrinogen/ml fibrinogen precursor solution (equivalent to 19.1 mg fibrinogen/ml fibrin formulation), 5.1 I.U. thrombin/ml thrombin precursor solution (equivalent to 0.067 I.U. thrombin/mg fibrinogen) and 1 μg TG-PDGF.AB/ml fibrin formulation (equivalent to 0.0262 μg TG-PDGF.AB/mg fibrinogen).
Fibrin Formulation 4: 38.2 mg fibrinogen/ml fibrinogen precursor solution (equivalent to 19.1 mg fibrinogen/ml fibrin formulation), 5.1 I.U. thrombin/ml thrombin precursor solution (equivalent to 0.067 I.U. thrombin/mg fibrinogen) and 11 μg TG-PDGF.AB/mL fibrin formulation (equivalent to 0.288 μg TG-PDGF.AB/ml fibrinogen)

Fibrin Formulation 5: 38.2 mg fibrinogen/ml fibrinogen precursor solution (equivalent to 19.1 mg fibrinogen/ml fibrin formulation), 2.55 I.U. thrombin/mL thrombin precursor solution (equivalent to 0.067 I.U. thrombin/mg fibrinogen) and 1 μg TG-PDGF.AB/mL fibrin formulation (equivalent to 0.0262 μg TG-PDGF.AB/ml fibrinogen)
Fibrin Formulation 6: 38.2 mg fibrinogen/mL fibrinogen precursor solution (equivalent to 19.1 fibrinogen/fibrin formulation, 2.55 I.U. thrombin/mL thrombin precursor solution) and 11 μg TG-PDGF.AB/mL fibrin formulation (equivalent to 0.288 TG-PDGF AB/mg fibrinogen)

The fibrin formulations gels were left drying for 1 hour at 37° C. They were subsequently incubated in 1 mL release buffer each at 37° C. The size of each gel was determined by weighing the tube containing buffer with and without the fibrin gel. At 2, 6, 24, 48 and 72 hours, the supernatants were collected and stored at −20° C. 1 mL fresh release buffer was added each time point. The PDGF concentrations in the supernatants were determined by ELISA, with all calculations performed and graphs plotted using Microsoft EXCEL.

Results

The results showed that at 6 hours between 4 and 7% of the PDGF was released from all the fibrin formulation samples. There was no remarkable differences of the release profile between the different samples The release afterwards up to 70 hours, i.e. before a fibrin matrix would start to be degraded in the body) was insignificant for all the samples (maximum another 2%). Low concentrations of TG PDGF AB are withheld in the matrix effectively within the ranges of the tested fibrinogen and thrombin concentrations.

### Example 7

### Pig Burn Model

Study Objective

The purpose of this study was to evaluate the adhesiveness and efficacy of several fibrin formulations and fibrin matrices by reduction of skin mesh graft dislocation and by reduction of mesh interstices.

Material & Methods

Female Yorkshire pigs were acclimatized for one week prior to surgery and fasted for 12 hours before surgery. To monitor contraction, large squares were tattooed around each wound site. Third degree contact burns were created on the animals, 5 on each flank. For this purpose a custom-made copper bar (4×4 cm², 160 g) was heated to 170° C. and placed on the sites for 20 sec without applying extra pressure (0.2 kg/cm² pressure).

Four days after burn wound creation, wound areas were traced on transparencies and the wounds were excised with an electrical dermatome to approximately 2.7 mm (i.e. full thickness). Wound sites were sprayed with 0.5 ml of fibrin formulation from a distance of 10-15 cm from the wound bed in a sweeping (painting) motion. Within 50 seconds wounds were covered with a meshed split thickness skin autograft (SSG) meshed in a 3:1 ratio (the skin graft was meshed to expand three times its original size) and a second layer of fibrin formulation of 0.5 ml thickness was applied over the whole wound area. The crosslinking of the fibrinogen converted the fibrin formulation into a fibrin matrix. The wounds were then covered by bandages.

Preparation of Test Samples

All fibrin matrices tested contained 1 μg/ml TG-PDGF.AB. The fibrinogen, thrombin and PDGF concentrations of the tested fibrin matrices are given in the table below:

| | Fibrinogen | Fibrinogen | Thrombin | | TG-PDGF.AB | |
|---|---|---|---|---|---|---|
| | (mg/ml | (mg/ml | (I.U./ml | Thrombin | (μg/ml | TG-PDGF.AB |
| | precursor | fibrin | precursor | (I.U./mg of | precursor | (μg/mg of |
| Sample | soln) | formulation) | solution) | fibrinogen) | solution) | fibrinogen) |
| control | 82.26 | 41.13 | 5.1 | 0.062 | 2 | 0.0243 |
| 1 | 56.2 | 28.1 | 3.4 | 0.06 | 2 | 0.036 |
| 2 | 42.13 | 21.1 | 2.55 | 0.06 | 2 | 0.047 |
| 3 | 42.13 | 21.1 | 5.1 | 0.12 | 2 | 0.047 |
| 4 | 28.1 | 14.01 | 5.1 | 0.18 | 2 | 0.071 |

On the day of operation, the device containing the precursor components were connected to the spray device for application. All preparations were carried out under aseptic conditions to minimize bioburden.

Evaluations

At postoperative days two, four, eight and fourteen, dressings were changed and digital images were taken. The following evaluations were done by observers or by digital image analysis:

1. Graft dislocation is the displacement of the skin autograft in comparison to the initial area and expressed as percentage of total effect size. This was scored on the living animal and was measured by using digital images.

2. Open wound area is the open mesh interstices in the remaining area within the total defect size after grafting. This was estimated on the living animal and was measured by using digital image analysis.

Statistics

Statistical analysis was performed with SPSS (Version 16.0 for MS Windows, SPSS Inc, Chicago, Ill.). The Mann-Whitney U test (MWU) was used to determine significant differences between the groups.

Results

Graft Dislocation

The grafts dislocation of all formulations indicated that all grafts had a low percentage of dislocation throughout the experiment. There was no statistical difference between the different formulations with regard to graft dislocation. This means that formulation 1 to 4, having lower concentration of fibrinogen, showed as good adherence of the skin graft throughout the experiment as the control formulation, which had a higher fibrinogen concentration.

Open Interstices (Wound Closure)

To determine if all wounds were equally covered, the areas covered by the split skin graft were measured using digital image analysis. Wound coverage on day 0 did not differ between treatments.

At 4 days after the grafting procedure, the percentage of open wound for the control formulation was 35±5%. A slightly lower open wound percentage was observed for the formulations 1 and 4 at 32±7% and 30±12% open interstices respectively. Formulations 2 and 3 had a slightly higher open wound percentage at 41±11% and 39±8% respectively. There was no statistical difference between all the formulations.

At 8 days after the grafting procedure, the open wound percentages were very low and there was no difference between the formulations tested.

We claim:

1. A fibrin formulation comprising:
(i) fibrinogen;
(ii) thrombin wherein the amount of thrombin is less than 0.3 UI of thrombin per mg of fibrinogen; and
(iii) an added bioactive factor in a concentration from about 0.0001 μg to about 1.5 μg per mg of fibrinogen.

2. The fibrin formulation of claim 1, further comprising a calcium source.

3. The fibrin formulation of claim 1, wherein the concentration of the added bioactive factor is in between about 0.0002 μg to about 0.8 μg bioactive factor per mg of fibrinogen.

4. The fibrin formulation of claim 1, wherein the concentration of added bioactive factor is in between about 0.0004 μg to about 0.5 μg bioactive factor per mg of fibrinogen.

5. The fibrin formulation of claim 1, wherein the concentration of the fibrinogen is in a range of between 7.5 to 30 mg fibrinogen per ml fibrin formulation.

6. The fibrin formulation of claim 1, wherein the concentration of the fibrinogen is in a range of between 10 to 29 mg fibrinogen per ml fibrin formulation.

7. The fibrin formulation of claim 1, wherein the concentration of the fibrinogen is in a range of between 15 to 27.5 mg fibrinogen per ml fibrin formulation.

8. The fibrin formulation of claim 1, wherein the added bioactive factor is a growth factor selected from the group consisting of members of the transforming growth factor (TGF β) superfamily, the platelet derived growth factor (PDGF) superfamily and the FGF superfamily.

9. The fibrin formulation of claim 8 wherein the growth factor is selected from the group consisting of PDGF AB, PDGF BB, PDGF D, TGFβ1, TGFβ3, VEGF 121, FGF 7 and IGF 1, most preferred PDGF AB.

10. The fibrin formulation of claim 1 wherein the added bioactive factor is a fusion protein comprising at least two domains, wherein the first domain comprises the growth factor and the second domain comprises a transglutaminase substrate domain.

11. The fibrin formulation of claim 10 wherein the second domain of the fusion protein comprises a Factor XIIIa substrate domain.

12. The fibrin formulation of claim 12, wherein the Factor XIIIa substrate domain comprises SEQ ID NO:1.

13. The fibrin formulation of claim 11, wherein the growth factor is selected from the group consisting of PDGF AB, PDGF BB, PDGF D, TGFβ1, TGFβ3, VEGF 121, FGF 7 and IGF 1, most preferred PDGF AB.

14. The fibrin formulation of claim 13, wherein the fusion protein comprises an amino acid sequence selected from the group consisting of SEQ ID NO:2 and SEQ ID NO:3.

15. A kit comprising:
(i) a first container comprising fibrinogen; and
(ii) a second container comprising thrombin, wherein the amount of thrombin is less than 0.3 I.U. thrombin per mg of fibrinogen,
(iii) added bioactive factor in a concentration range of between about 0.0001 μg to about 1.5 μg bioactive factor to per mg of fibrinogen contained in the first and/or second container or in a separate third container.

16. The kit of claim 15, wherein the concentration of the added bioactive factor is in a range of between about 0.0004 to about 0.5 μg bioactive factor per mg of fibrinogen.

17. The kit of claim 15, wherein the fibrinogen concentration is in a range of between 10 to 29 mg fibrinogen per ml fibrin formulation.

18. The kit of claim 15, wherein the added bioactive factor is a growth factor selected from the group consisting of PDGF AB, PDGF BB, PDGF D, TGFβ1, TGFβ3, VEGF 121, FGF 7 and IGF 1, most preferred PDGF AB.

19. The kit of claim 15, wherein the bioactive factor is a fusion protein comprising at least two domains, wherein the first domain comprises the growth factor and the second domain comprises a transglutaminase substrate domain.

20. A method for preparing a fibrin formulation comprising the steps of:
(i) providing a fibrinogen solution;
(ii) providing a thrombin solution wherein the amount of thrombin is less than 0.3 I.U. thrombin per mg of fibrinogen;
(iii) providing at least one added bioactive factor in a concentration range of between about 0.0001 μg to about 1.5 μg bioactive factor per mg of fibrinogen.


SEQUENCE LISTING (3 sequences)

SEQ ID NO: 1 - protein, 7 aa, Artificial Sequence
  Synthetic N terminal domain of alpha 2 plasmin inhibitor
NQEQVSP

SEQ ID NO: 2 - protein, 131 aa, Artificial Sequence
  Synthetic TG-PDGF A
MNQEQVSPLPVELPLIKMKPHSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 3 - protein, 130 aa, Artificial Sequence
  Synthetic TG-PDGF B
MNQEQVSPLPVELPLIKMKPHSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVT